(19) United States
(12) Patent Application Publication
(10) Pub. No.: US 2012/0004551 A1
(43) Pub. Date: Jan. 5, 2012

(54) Ultrasonic diagnosis apparatus and ultrasonic diagnosis method

(75) Inventor: Kimito Katsuyama, Ashigarakami-gun (JP)

(73) Assignee: FUJIFILM Corporation, Tokyo (JP)

(21) Appl. No.: 13/067,866

(22) Filed: Jun. 30, 2011

(30) Foreign Application Priority Data
Jun. 30, 2010 (JP) 2010-150064

(51) Int. Cl. A61B 8/14

(52) U.S. Cl. 600/443

(57) ABSTRACT

An ultrasonic diagnosis method of one aspect of the presently disclosed subject matter: transmitting ultrasonic waves to an object through an ultrasonic probe including a plurality of ultrasonic transducers, and outputting ultrasonic detection signals after receiving the ultrasonic waves reflected by the object; calculating at least one of a reception time and a reception wave at each of respective elements corresponding to two or more lattice points at different depths in the object with respect to the ultrasonic probe; and calculating a local sound velocity which is a sound velocity in a local region, using calculated one of the reception time and the reception wave.

### BACKGROUND OF THE INVENTION

1. Field of the Invention

The presently disclosed subject matter generally relates to an ultrasonic diagnosis apparatus and an ultrasonic diagnosis method, and more particularly, to an ultrasonic diagnosis apparatus and an ultrasonic diagnosis method which are used to capture and display ultrasonic images of an object with the use of ultrasonic waves, and are used in diagnosing tissues.

2. Description of the Related Art

Conventionally, a sound velocity value in a portion (a diagnosed site) in an object (such a sound velocity value will be hereinafter referred to as a local sound velocity value) is measured with the use of ultrasonic waves. For example, the following methods have been suggested. According to one of the methods, two oscillators for transmission and reception are positioned to face each other, and a sound velocity value in an object is calculated from the distance between the oscillators and a time period for propagation of ultrasonic waves between the oscillators. According to another one of the methods, two sets of oscillators arranged at predetermined intervals are used for transmission and reception, and a propagation velocity is calculated from a time period for ultrasonic propagation between the oscillators, transmission and reception angles, and the distances between the oscillators in the respective pairs.

Japanese Patent Application Laid-Open No. 5-95946 discloses the following method of measuring values of local sound velocities. According to Japanese Patent Application Laid-Open No. 5-95946, ultrasonic waves are transmitted while the output angle from a transmission oscillator toward an object is being changed (varied), and ultrasonic waves are received while the incident angle is being varied by a reception oscillator. All the periods of time elapsed from transmission to reception are stored into a memory. A hypothetical distribution of sound velocities is set, and, based on the distribution of sound velocities, the elapsed time is calculated for each of the output angles and incident angles. The hypothetical distribution of sound velocities is then corrected so that the differences between the calculated values of period of elapsed time and actual measurement values are minimized. The sound velocity value in the object is then determined from the resultant distribution of sound velocities.

### SUMMARY OF THE INVENTION

A sound velocity value V in an object OBJ1 made of a medium having a constant sound velocity value can be calculated in the following manner. As illustrated in FIG. 10A, where L represents the distance from a reflection point (region) X1_{ROI} in the object OBJ1 to an ultrasonic probe 300A, the period of time T elapsed from reflection of ultrasonic waves at the reflection point X1_{ROI} to reception of the ultrasonic waves by an element 302A₀ located immediately below the reflection point X1_{ROI} is expressed as T=L/V. Where the period of time elapsed before reception by an element 302A₁ located at a distance X in the X-direction (the array direction of elements 302A) from the element 302A₀ is expressed as T+ΔT, the delay time ΔT between the elements 302A₀ and 302A₁ is expressed by the following mathematical formula (1):

[Formula 1]

ΔT=ΔL/V(where ΔL=\sqrt{L²+X²}−L) (1)

Therefore, after the ultrasonic waves are reflected at the reflection point X1_{ROI} the time T after the transmission of the ultrasonic waves, the periods of time 2T and 2T+ΔT elapsed before the ultrasonic waves are received by the element 302A₀ and another element, respectively, are measured. In this manner, the distance L to the reflection point X1_{ROI} and the velocity V can be uniquely determined.

Where the ultrasonic waves reflected from the reflection point X1_{ROI} can be clearly recognized, the distance L and the velocity V can be determined from the elapsed time measured at the element 302A₀ and another element. However, ultrasonic detection signals which are output from the respective elements 302A are normally resulted from interferences by signals from numerous reflection points, and it is difficult to distinguish only the signals supplied from a specific reflection point. Therefore, in practice, the distance L to the reflection point X1_{ROI}, the delay time ΔT, and the sound velocity value V are uniquely determined from the spatial frequency, sharpness, and contrast in a re-constrcted image in a region of interest in the vicinity of the reflection point X1_{ROI}.

As described above, where the sound velocity in an object is uniform (constant), the value of the sound velocity can be calculated by the above method. Where the internal sound velocity is not uniform as in an object OBJ2 illustrated in FIG. 10B, however, it is difficult to calculate the distance L to a reflection point (region) X2_{ROI} and the sound velocity values V and V′ by the above described method.

To counter this problem, the inventor suggested a method of determining a local sound velocity where the sound velocity in an object is not uniform (Japanese Patent Application Laid-Open No. 2010-99452). According to this method, a hypothetical sound velocity is set in a region of interest in an object, and an optimum sound velocity value at a lattice point (grid point) set in a shallower region than the region of interest is set. Based on the hypothetical sound velocity and the optimum sound velocity value, an image of the region of interest is generated from reception signals of the respective elements obtained when ultrasonic waves are transmitted to the region of interest, and the image of the region of interest is then analyzed. Alternatively, an optimum sound velocity value at a representative lattice point or a reception wave at a representative lattice point in the region of interest is calculated. The local sound velocity value in the region of interest is then determined by comparing the hypothetical sound velocity in the region of interest with the optimum sound velocity value or the reception wave calculated based on the optimum sound velocity value set in the shallower region than the region of interest. As described above, the method suggested previously by the present applicant is a method of approximating the reception wave at each lattice point by the ambient sound velocity (an optimum sound velocity), and this method enables measurements of local sound velocities even where the sound velocity in the object is not uniform.

Where the unsteadiness of the sound velocity in the object is higher than predicted, however, the above described method of approximating the reception wave at each lattice point by the ambient sound velocity cannot cope with the unsteadiness of the sound velocity.

The presently disclosed subject matter has been made in view of the above circumstances, and the object thereof is to provide an ultrasonic diagnosis apparatus (ultrasonic image processing apparatus) and an ultrasonic diagnosis method (ultrasonic image processing method) which can determine a local sound velocity with high precision even where the sound velocity in an object is not uniform, and the reception time at each lattice point cannot be approximated by the ambient sound velocity.

To achieve the above object, a first aspect of the presently disclosed subject matter provides an ultrasonic diagnosis apparatus including: an ultrasonic probe which includes a plurality of ultrasonic transducers configured to transmit ultrasonic waves to an object, to receive the ultrasonic waves reflected by the object, and to output ultrasonic detection signals; a reception time calculating device configured to calculate at least one of a reception time and a reception wave at each of respective elements corresponding to two or more lattice points at different depths in the object with respect to the ultrasonic probe; and a local sound velocity calculating device configured to calculate a local sound velocity which is a sound velocity in a local region, using calculated one of the reception time and the reception wave.

Accordingly, a local sound velocity is calculated by determining the reception time at each lattice point, instead of the ambient sound velocity. Accordingly, the local sound velocity can be calculated with high precision even where the sound velocity in the object is not uniform.

In a second aspect of the presently disclosed subject matter, the two or more lattice points at different depths with respect to the ultrasonic probe include a lattice point in a region of interest in the object and a lattice point which is set in a shallower region than the region of interest.

In a third aspect of the presently disclosed subject matter, the local sound velocity calculating device calculates the local sound velocity by comparing a reception wave at the region of interest with a resultant reception wave formed by combining a reception wave at the lattice point set in the shallower region with a delay determined by a hypothetical sound velocity hypothetically set in the region of interest.

Accordingly, a local sound velocity can be calculated with high precision even where the sound velocity in the object is not uniform.

In a fourth aspect of the presently disclosed subject matter, the local sound velocity calculating device calculates the local sound velocity by comparing a reception time at the lattice point of the region of interest with a smallest value of sums, each of the sums being a sum of a propagation time determined based on a hypothetical sound velocity hypothetically set in the region of interest and the reception time at the lattice point set in the shallower region, the propagation time being a period for propagation from the lattice point in the region of interest to the lattice point set in the shallower region.

Accordingly, a local sound velocity can be calculated with high precision even where the sound velocity in the object is not uniform.

Also, to achieve the above object, a fifth aspect of the presently disclosed subject matter provides an ultrasonic diagnosis apparatus including: an ultrasonic probe which includes a plurality of ultrasonic transducers configured to transmit ultrasonic waves to an object, to receive the ultrasonic waves reflected by the object, and to output ultrasonic detection signals; a reception time calculating device configured to calculate at least one of a reception time and a reception wave at each of respective elements corresponding to two or more lattice points at different depths in the object with respect to the ultrasonic probe; and a local reception time calculating device configured to calculate a local reception time which is a reception time in a local region, using calculated one of the reception time and the reception wave.

Accordingly, a local reception time, instead of a local sound velocity, can be calculated even where the sound velocity in the region of interest is not uniform.

In a sixth aspect of the presently disclosed subject matter, the two or more lattice points at different depths with respect to the ultrasonic probe include a lattice point in a region of interest in the object and a lattice point set in a shallower region than the region of interest.

In a seventh aspect of the presently disclosed subject matter, the local reception time calculating device calculates a local reception time by regarding the lattice point set in the shallower region as a virtual element, matching and adding up a reception wave at the lattice point in the region of interest and a delay which is a reception time at the lattice point set in the shallower region, and carrying out a phase aberration analysis on a reception signal of the virtual element.

Accordingly, a local reception time, instead of a local sound velocity, can be calculated even where the sound velocity in the region of interest is not uniform.

In an eighth aspect of the presently disclosed subject matter, the local reception time calculating device regards the lattice point set in the shallower region as a virtual element, and calculates a local reception time at the virtual element, a local reception time being a latest time among times each obtained by subtracting a delay which is a reception time at the lattice point set in the shallower portion from the reception time at the lattice point in the region of interest.

Accordingly, a local reception time, instead of a local sound velocity, can be calculated even where the sound velocity in the region of interest is not uniform.

Also, to achieve the above object, a ninth aspect of the presently disclosed subject matter provides an ultrasonic diagnosis method including: transmitting ultrasonic waves to an object through an ultrasonic probe including a plurality of ultrasonic transducers, and outputting ultrasonic detection signals after receiving the ultrasonic waves reflected by the object; calculating at least one of a reception time and a reception wave at each of respective elements corresponding to two or more lattice points at different depths in the object with respect to the ultrasonic probe; and calculating a local sound velocity which is a sound velocity in a local region, using calculated one of the reception time and the reception wave.

By this method, a local sound velocity is calculated by determining the reception time at each lattice point, instead of the ambient sound velocity. Accordingly, the local sound velocity can be calculated with high precision even where the sound velocity in the object is not uniform.

In a tenth aspect of the presently disclosed subject matter, the calculating the reception time includes using an image analysis and a phase aberration analysis to calculate the reception time.

In an eleventh aspect of the presently disclosed subject matter, the two or more lattice points at different depths with respect to the ultrasonic probe include a lattice point in a region of interest in the object and a lattice point which is set in a shallower region than the region of interest.

In a twelfth aspect of the presently disclosed subject matter, the calculating the local sound velocity includes comparing the reception wave in the region of interest with a resultant reception wave formed by combining the reception wave at the lattice point set in the shallower region with a delay which is determined by a hypothetical sound velocity hypothetically set in the region of interest.

By this method, a local sound velocity can be calculated with high precision even where the sound velocity in the object is not uniform.

In a thirteenth aspect of the presently disclosed subject matter, the calculating the local sound velocity includes comparing a reception time at the lattice point in the region of interest with a smallest value of sums, each of the sums being a sum of a propagation time determined based on a hypothetical sound velocity hypothetically set in the region of interest and a reception time at the lattice point set in the shallower region, the propagation time being a period for propagation from the lattice point in the region of interest to the lattice point set in the shallower region.

To also achieve the above object, a fourteenth aspect of the presently disclosed subject matter provides an ultrasonic diagnosis method including: transmitting ultrasonic waves to an object through an ultrasonic probe including a plurality of ultrasonic transducers, and outputting ultrasonic detection signals after receiving the ultrasonic waves reflected by the object; calculating at least one of a reception time and a reception wave at each of respective elements corresponding to two or more lattice points at different depths in the object with respect to the ultrasonic probe; and calculating a local reception time which is a reception time in a local region, using calculated one of the reception time and the reception wave.

By this method, a local reception time, instead of a local sound velocity, can be calculated even where the sound velocity in the region of interest is not uniform.

In a fifteenth aspect of the presently disclosed subject matter, the calculating the reception time includes using an image analysis and a phase aberration analysis to calculate the reception time.

In a sixteenth aspect of the presently disclosed subject matter, the two or more lattice points at different depths with respect to the ultrasonic probe include a lattice point in a region of interest in the object and a lattice point which is set in a shallower region than the region of interest.

In a seventeenth aspect of the presently disclosed subject matter, the calculating the local reception time includes regarding the lattice point set in the shallower region as a virtual element, matching and adding up a reception wave at the lattice point in the region of interest and a delay which is a reception time at the lattice point set in the shallower region, and carrying out a phase aberration analysis on a reception signal of the virtual element.

By this method, a local reception time, instead of a local sound velocity, can be calculated even where the sound velocity in the region of interest is not uniform.

In an eighteenth aspect of the presently disclosed subject matter, the calculating the local reception time includes regarding the lattice point set in the shallower region as a virtual element, and calculating a local reception time at the virtual element, a local reception time being a latest time among times each obtained by subtracting a delay which is a reception time at the lattice point set in the shallower portion from a reception time at the lattice point in the region of interest.

By this method, a local reception time, instead of a local sound velocity, can be calculated even where the sound velocity in the region of interest is not uniform.

As described above, according to the presently disclosed subject matter, a local sound velocity is calculated by determining one of the reception time and the reception wave at each of the elements corresponding to respective lattice points at respective lattice points, instead of determining the ambient sound velocity. Accordingly, a local sound velocity can be calculated with high precision even where the sound velocity in the object is not uniform. Also, a local reception time, instead of a local sound velocity, can be calculated even where the sound velocity in the region of interest is not uniform.

### BRIEF DESCRIPTION OF THE DRAWINGS

FIG. 1 is a block diagram schematically illustrating a structure of an ultrasonic diagnosis apparatus according to an embodiment of the presently disclosed subject matter;

FIG. 2 is a flowchart illustrating an operation according to a first embodiment of the presently disclosed subject matter;

FIG. 3 is a schematic view illustrating an operation to calculate a value of a local sound velocity according to the first embodiment;

FIG. 4 is a flowchart illustrating an operation according to a second embodiment of the presently disclosed subject matter;

FIG. 5 is a schematic view illustrating an operation to calculate a value of a local sound velocity according to the second embodiment of the presently disclosed subject matter;

FIG. 6 is a flowchart illustrating an operation according to a third embodiment of the presently disclosed subject matter;

FIG. 7 is a flowchart illustrating an operation according to a fourth embodiment of the presently disclosed subject matter;

FIG. 8 is a schematic view illustrating a situation where a delay is subtracted from the reception time at each element in the region of interest in the fourth embodiment;

FIG. 9 is a diagram for explaining a situation where delays are subtracted from reception waves in the fourth embodiment; and

FIGS. 10A and 10B are an explanatory views schematically illustrating an operation to calculate the value of a local sound velocity.

### DETAILED DESCRIPTION OF THE PREFERRED EMBODIMENTS

The following is a detailed description of ultrasonic diagnosis apparatuses and ultrasonic diagnosis methods according to the presently disclosed subject matter, with reference to the accompanying drawings.

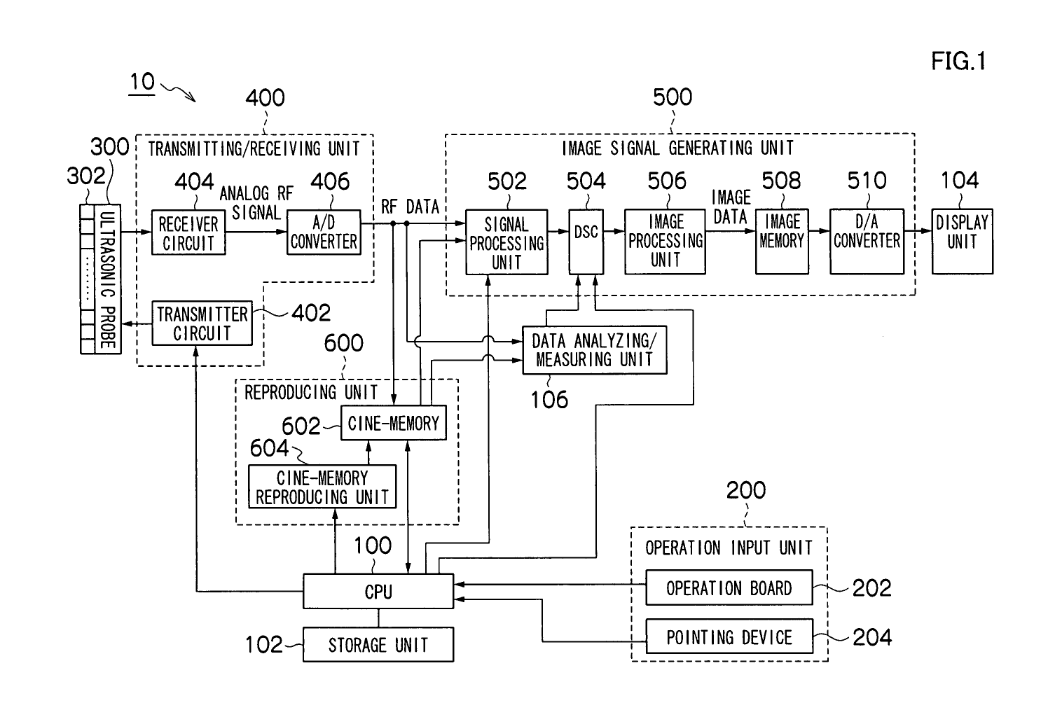

FIG. 1 is a block diagram schematically illustrating a structure of an ultrasonic diagnosis apparatus according to an embodiment of the presently disclosed subject matter.

As illustrated in FIG. 1, the ultrasonic diagnosis apparatus 10 of this embodiment transmits ultrasonic beams from an ultrasonic probe 300 to an object OBJ, receives ultrasonic beams reflected by the object OBJ (ultrasonic echoes), and creates and displays an ultrasonic image based on a ultrasonic echo detection signal.

A CPU (Central Processing Unit) 100 controls respective blocks of the ultrasonic diagnosis apparatus 10, in accordance with operations input through an operation input unit 200.

The operation input unit 200 is an input device which receives operations input from an operator. The operation input unit 200 includes an operation board 202 and a pointing device 204. The operation board 202 includes a keyboard which receives inputs of textual information (patient information, for example), a display mode switching button for issuing an instruction to switch a display mode between a mode for displaying only an amplitude image (a B-mode image) and a mode for displaying a result of determination made on a value of the local sound velocity, a freeze button for issuing an instruction to switch a mode between a live mode and a frozen mode, a cine-memory reproduction button for issuing an instruction to reproduce a cine-memory, and an analysis/measurement button for issuing an instruction to analyze/measure an ultrasonic image. The pointing device 204 receives inputs of designation of a region on a screen of a display unit 104. A trackball or a mouse may be used as the pointing device 204, for example. Alternatively, a touch panel may be used as the pointing device 204, for example.

A storage unit 102 is a storage device which stores a control program for the CPU 100 to control the respective blocks of the ultrasonic diagnosis apparatus 10, and may be a hard disk or a semiconductor memory, for example.

A display unit 104 may be a CRT (Cathode Ray Tube) display or a liquid crystal display. A display unit 104 is configured to display an ultrasonic image (a video image or a still image) and various kinds of setting screens.

The ultrasonic probe 300 is a probe which is in contact with object OBJ when used. The ultrasonic probe 300 includes ultrasonic transducers 302 which constitute a one- or two-dimensional transducer array. The ultrasonic transducers 302 transmit ultrasonic beams to the object OBJ, based on a drive signal applied from a transmitter circuit 402. The ultrasonic transducers 302 then receive ultrasonic echoes reflected from the object OBJ, and output detection signals.

The ultrasonic transducers 302 include oscillators. Each of the oscillators is formed by providing electrodes at both ends of a material having piezoelectric properties (a piezoelectric material). Examples of piezoelectric materials for forming such oscillators include piezoelectric ceramics such as PZT (Pb (lead) zirconate titanate) and polymer piezoelectric element such as PVDF (polyvinylidene difluoride). When an electric signal is sent to the electrodes of the oscillators, the piezoelectric material expands and contracts, and ultrasonic waves are generated in each oscillator by the expansion and contraction of the piezoelectric material. For example, where a pulsed electric signal is sent to the electrodes of the oscillators, pulsed ultrasonic waves are generated. Where a continuous-wave electric signal is sent to the electrodes of the oscillators, continuous ultrasonic waves are generated. The ultrasonic waves generated in the respective oscillators are combined to form an ultrasonic beam. Also, when the ultrasonic waves are received by the respective oscillators, the piezoelectric material of each oscillator expands and contracts, to generate an electric signal. The electric signals generated in the respective oscillators are output as ultrasonic detection signals to a receiver circuit 404.

It should be noted that two or more kinds of elements of different ultrasonic transduction types can be used as the ultrasonic transducers 302. For example, oscillators formed by the above described piezoelectric material may be used as elements which transmit ultrasonic waves, and ultrasonic transducers of a photodetection type may be used as the elements which receive ultrasonic waves. Here, the ultrasonic transducers of a photodetection type perform a detecting operation by converting an ultrasonic signal into an optical signal, and such ultrasonic transducers may be Fabry-Perot resonators or fiber Bragg gratings, for example.

Next, an ultrasonic diagnosing process to be performed in the live mode is described. The live mode is a mode in which an ultrasonic image (a video image) obtained by bringing the ultrasonic probe 300 into contact with the object OBJ and performing transmission/reception of ultrasonic waves is displayed and analyzed/measured.

The ultrasonic probe 300 is brought into contact with the object OBJ, and ultrasonic diagnosing is started in accordance with an instruction input through the operation input unit 200. Then, the CPU 100 outputs a control signal to a transmitting/receiving unit 400, to cause the transmitting/receiving unit 400 to start transmission of ultrasonic beams to the object OBJ and reception of ultrasonic echoes from the object OBJ. The CPU 100 sets an ultrasonic beam transmitting direction and an ultrasonic echo receiving direction for each ultrasonic transducer 302.

Further, the CPU 100 selects a transmission delay pattern in accordance with the ultrasonic beam transmitting direction, and selects a reception delay pattern in accordance with the ultrasonic echo receiving direction. Here, a transmission delay pattern is delay time pattern data to be supplied to the drive signal, so as to form ultrasonic beams in a desired direction with ultrasonic waves transmitted from two or more ultrasonic transducers 302. A reception delay pattern is delay time pattern data to be received by two or more ultrasonic transducers 302. Such transmission delay patterns and reception patterns are stored beforehand into the storage unit 102. The CPU 100 selects a transmission delay pattern and a reception delay pattern from those stored in the storage unit 102. In accordance with the selected transmission delay pattern and reception delay pattern, the CPU 100 outputs the control signal to the transmitting/receiving unit 400, to control transmission and reception of ultrasonic waves.

The transmitter circuit 402 generates the drive signal in accordance with the control signal from the CPU 100, and applies the drive signal to the ultrasonic transducers 302. At this point, based on the transmission delay pattern selected by the CPU 100, the transmitter circuit 402 delays the drive signal to be applied to each of the ultrasonic transducers 302. Here, the transmitter circuit 402 performs a transmit focusing process to adjust (delay) the timing to apply the drive signal to each of the ultrasonic transducers 302, so that the ultrasonic waves transmitted from the respective ultrasonic transducers 302 form ultrasonic beams. Alternatively, the timing to apply the drive signal may be adjusted so that the ultrasonic waves simultaneously transmitted from two or more ultrasonic transducers 302 reach the entire imaging region of the object OBJ.

The receiver circuit 404 receives and amplifies the ultrasonic detection signals output from the respective ultrasonic transducers 302. As described above, the distances between the respective ultrasonic transducers 302 and the ultrasonic reflection source X_{ROI} in the object OBJ vary. Therefore, the periods of time required for a reflected wave to reach the respective ultrasonic transducers 302 vary. The receiver circuit 404 includes a delay circuit. In accordance with a sound velocity (hereinafter referred to as a hypothetical sound velocity) or the distribution of velocities of sound which are set based on the reception delay pattern selected by the CPU 100, the receiver circuit 404 delays each detection signal by the amount equivalent to the difference (delay time) in arrival time of the reflected wave. The receiver circuit 404 then performs a reception focusing operation by matching and adding up the detection signals having the delay times added thereto. If another ultrasonic reflection source exists at a different location from the ultrasonic reflection source X_{ROI}, the arrival times of ultrasonic detection signals from the other ultrasonic reflection source differ from those of the ultrasonic detection signals from the ultrasonic reflection source X_{ROI}. Therefore, the phases of the ultrasonic detection signals from the other ultrasonic reflection source are cancelled by adding up the detection signals at the adding circuit of the receiver circuit 404. In this manner, the reception signal from the ultrasonic reflection source X_{ROI} becomes the largest, and focusing is achieved. Through the above reception focusing operation, an acoustic ray signal (hereinafter referred to as the RF signal) with ultrasonic echoes having the narrowed focuses is formed.

The A/D converter 406 converts the analog RF signal output from the receiver circuit 404 into a digital RF signal (hereinafter referred to as RF data). Here, the RF data contains phase information about reception waves (carrier waves). The RF data output from the A/D converter 406 is input to a signal processing unit 502 and a cine-memory 602.

The cine-memory 602 sequentially stores the RF data input from the A/D converter 406. The cine-memory 602 also stores and associates information about frame rates input from the CPU 100 (parameters indicating a depth of a reflection location of ultrasonic waves, a scanning line density, and a width of a field of view, for example) with the above described RF data.

For the RF data, the signal processing unit 502 performs STC (Sensitivity Time gain Control) to correct attenuations caused by distances in accordance with the depth of the reflection location of ultrasonic waves. After that, the signal processing unit 502 performs an envelope detection process to generate B-mode image data (image data in which amplitudes of ultrasonic echoes are represented by brightness (luminance) of dots).

The B-mode image data generated by the signal processing unit 502 is obtained by a different scanning technique from a conventional television signal scanning technique. Therefore, a DSC (Digital Scan Converter) 504 converts (raster-converts) the B-mode image data into conventional image data (image data according to the television signal scanning system (the NTSC (National Television System Committee) system), for example). An image processing unit 506 performs various kinds of necessary image processing (gradation processing, for example) on the image data input from the DSC 504.

The image memory 508 stores the image data input from the image processing unit 506. A D/A converter 510 converts the image data read from the image memory 508 into an analog image signal, and outputs the analog image signal to the display unit 104. In this manner, an ultrasonic image (a video image) captured by the ultrasonic probe 300 is displayed on the display unit 104.

In this embodiment, detection signals subjected to the reception focusing operation at the receiver circuit 404 are RF signals. However, detection signals having not been subjected to a reception focusing operation may be used as RF signals. In that case, the ultrasonic detection signals output from the ultrasonic transducers 302 are amplified at the receiver circuit 404, and the amplified detection signals or the RF signals are A/D converted by the A/D converter 406 to generate RF data. The RD data is supplied to the signal processing unit 502, and is also stored into the cine-memory 602. The reception focusing operation is digitally performed at the signal processing unit 502.

Next, the cine-memory reproduction mode is described. The cine-memory reproduction mode is a mode in which an ultrasonic diagnosis image is displayed and analyzed/measured, based on the RF data stored in the cine-memory 602.

When a cine-memory reproduction button of the operation board 202 is pressed, the CPU 100 switches the operation mode of the ultrasonic diagnosis apparatus 10 to the cine-memory reproduction mode. In the cine-memory reproduction mode, the CPU 100 instructs a cine-memory reproducing unit 604 to reproduce RF data designated by an operation input from an operator. Based on the instruction from the CPU 100, the cine-memory reproducing unit 604 reads the RF data from the cine-memory 602, and transmits the RF data to the signal processing unit 502 of the image signal generating unit 500. The RF data transmitted from the cine-memory 602 is subjected to predetermined processing (the same processing as that performed in the live mode) at the signal processing unit 502, the DSC 504, and the image processing unit 506, and is converted into image data. The image data is then output to the display unit 104 via the image memory 508 and the D/A converter 501. In this manner, the ultrasonic image (a video image or a still image) based on the RF data stored in the cine-memory 602 is displayed on the display unit 104.

In either one of the live mode and the cine-memory reproduction mode, when the freeze button of the operation board 202 is pressed while an ultrasonic image (a video image) is being displayed, the ultrasonic image being displayed at the time of the pressing of the freeze button is turned into a still image on the display unit 104. Accordingly, the operator can observe the still image displayed in the region of interest (ROI).

When the analysis/measurement button of the operation board 202 is pressed, an analyzing/measuring operation designated by an operation input from the operator is performed. When the analysis/measurement button is pressed in each operation mode, a data analyzing/measuring unit 106 obtains RF data having not subjected to image processing from the A/D converter 406 or the cine-memory 602, and performs the analyzing/measuring operation (an analysis of deformation of a tissue site (hardness diagnosing), a measurement of a bloodstream, a measurement of movement of a tissue site, or a measurement of an IMT (Intima-Media Thickness) value, for example) designated by the operator with the use of the RF data. The data analyzing/measuring unit 106 also performs an operation to measure a value of a local sound velocity, which will be described later in detail. The results of the analysis/measurement carried out by the data analyzing/measuring unit 106 are output to the DSC 504 of the image signal generating unit 500. The DSC 504 inserts the analysis/measurement results from the data analyzing/measuring unit 106 into the image data of an ultrasonic image, and outputs the image data having the results inserted thereinto to the display unit 104. In this manner, the ultrasonic image and the analysis/measurement results are displayed on the display unit 104.

When the display mode switching button is pressed, the display mode is switched between a mode to display only a B-mode image, a mode to superimpose the result of determination of the value of a local sound velocity upon the B-mode image (a display which changes color coding or luminance in accordance with the value of the local sound velocity, or a display which connects dots having the same value of the local sound velocity), and a mode to display the B-mode image and an image of the result of determination of the value of the local sound velocity which are arranged in a display screen. Accordingly, the operator can spot a pathological lesion by observing the result of determination of the value of the local sound velocity.

Based on the result of determination of the value of the local sound velocity, at least one of a transmit focus operation and a reception focusing operation may be performed to obtain a B-mode image, and the B-mode image may be displayed on the display unit 104.

In the following, the functions of the ultrasonic diagnosis apparatus 10 of this embodiment are described.

The presently disclosed subject matter enables a measurement of a local sound velocity even where the sound velocity in an object is not uniform and the reception time (or reception wave) at each of the elements corresponding to respective lattice points cannot be approximated by the ambient sound velocity. According to the presently disclosed subject matter, a local reception time, instead of a local sound velocity, can be determined even where the sound velocity in the region of interest is not uniform.

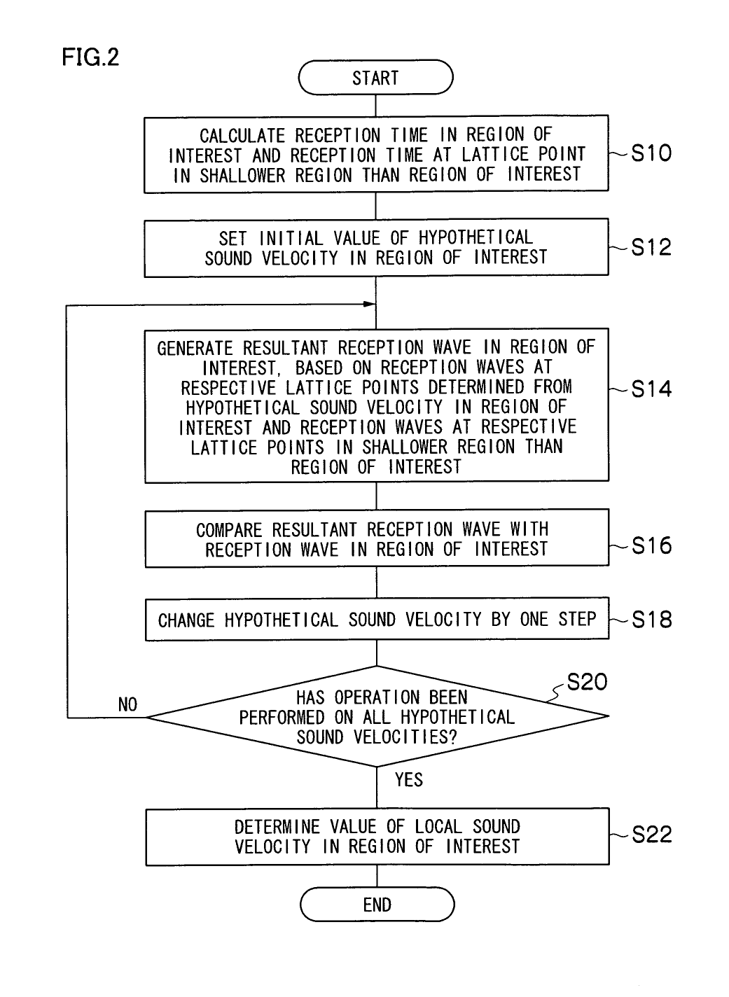

In the following, referring first to the flowchart illustrated in FIG. 2, a first embodiment is described. The first embodiment enables a measurement of a local sound velocity even where the sound velocity in the object is not uniform and the reception time (or reception wave) of each of the elements corresponding to the respective lattice points cannot be approximated by the ambient sound velocity. In the following embodiment, to determine the local sound velocity of a region of interest or reception time of the ultrasonic wave at the region of interest with the use of the reception time or reception wave at each of the elements corresponding to the respective lattice points, the reception time at each element is determined, and the reception wave at each of the elements is then determined as needed. Alternatively, the reception wave at each of the elements may be first determined, and the reception time at each of the elements may be then determined as needed. To determine the reception wave at each of the elements, scattering is reduced by subjecting each of the lattice points to a transmit focus, and only the signal formed by receiving the reflection from each of the lattice point should be used.

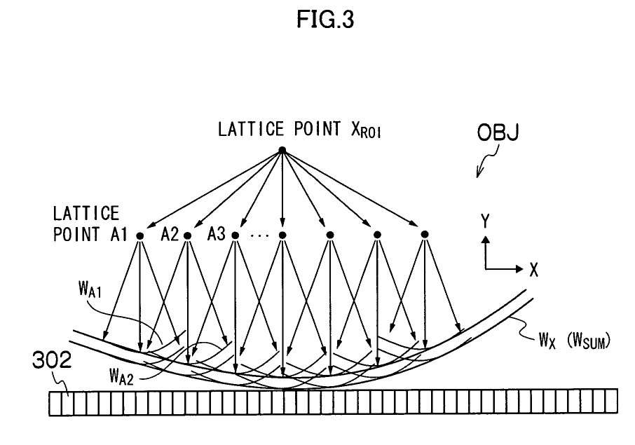

FIG. 3 schematically illustrates an operation to calculate the value of a local sound velocity according to this embodiment.

As illustrated in FIG. 3, a representative lattice point in a region of interest ROI in the object OBJ is represented by X_{ROI}, and the lattice points arranged at regular intervals in the X-Y direction in shallower positions than the lattice point X_{ROI} (or in positions close to the ultrasonic transducers 302, or in a region between the lattice point X_{ROI} and the ultrasonic transducers 302) are represented by A1, A2, A3, . . . The sound velocities at least between the lattice point X_{ROI} and the respective lattice points A1, A2, A3, . . . are assumed to be uniform. Here, the range of lattice points A1, A2, A3, . . . (a region where the lattice points A1, A2, A3, . . . are arranged) and the number of lattice points A1, A2, A3, . . . to be used in the operation to determine the value of the local sound velocity in the region of interest are determined in advance.

First, in step S10 of FIG. 2, the reception time in the region of interest and the reception times at the lattice points A1, A2, A3, . . . located in a shallower region than the region of interest are calculated. The reception time in the region of interest or at the lattice point X_{ROI}, and the reception times at the respective lattice points A1, A2, and A3, . . . can be calculated by a known image analysis method and a known phase aberration analysis method.

The image analysis method used herein is a method of hypothetically setting the mean sound velocity (and depth) and determining such a value that the characteristics such as the sharpness and contrast of the image of the sound source are maximized, as disclosed in Japanese Patent Application Laid-Open No. 2007-2045 and the like.

The phase aberration analysis method is disclosed in Japanese Patent Application Laid-Open No. 6-105841 and the like. According to this method, a signal is set as a reference signal for reception signals of the respective elements of an ultrasonic probe, and the phase differences between the reference signal and the reception signals are detected. The phase difference detection results of each two adjacent elements (neighboring elements) are compared, and the difference between the results is represented by D. In a graph which plots the number of elements of the ultrasonic transducer 302 on the abscissa axis, and plots the phase differences between the reference signal S and the reception signals of the respective elements on the ordinate axis, 360 degrees is added at discontinuous points from positive to negative (or when the above described difference D is smaller than −180 degrees), and 360 degrees is subtracted at discontinuous points from negative to positive (or when the above described difference D is larger than 180 degrees). In this manner, discontinuous curves are turned into continuous curves, and thus, the phase aberration in a wide region is detected with high precision.

In step S12 of FIG. 2, the initial value of a hypothetical sound velocity in the region of interest is set.

In step S14, a resultant reception wave at the lattice point X_{ROI} is formed by overlapping the reception waves of the respective lattice points on one another with the use of delays determined by the hypothetical sound velocity in the region of interest.

That is, the resultant reception wave W_{SUM} from the lattice point X_{ROI} is formed with the use of the reception waves W_{A1}, W_{A2}, . . . from the lattice points A1, A2, A3, . . . and the delays obtained from the hypothetical sound velocity, as illustrated in FIG. 3.

In step S16, the resultant reception wave W_{SUM} is compared with the reception wave W_{X} from the region of interest determined from the reception time in the region of interest determined in step S10. If the difference between the resultant reception wave W_{SUM} and the reception wave W_{X} compared this time is smaller than the difference between the resultant reception wave W_{SUM} and the reception wave W_{X} from the region of interest used in the previous comparison, the resultant reception wave W_{SUM} (or the hypothetical sound velocity corresponding to the resultant reception wave W_{SUM}) is stored.

In step S18, the hypothetical velocity is changed by one step. In step S20, a determination is made to determine whether the operation has been performed on all the hypothetical sound velocities. It should be noted that the range of hypothetical sound velocities to be subjected to the operation is determined in advance.

If the operation has not been performed on all the hypothetical sound velocities, the operation returns to step S14, and the above described procedures are repeated.

If the operation has been performed on all the hypothetical sound velocities, the operation moves on to step S22, and the value of the local sound velocity in the region of interest is calculated. Specifically, the resultant reception wave W_{SUM} stored last time as a result of the repetition of the above procedures (or the hypothetical sound velocity corresponding to this resultant reception wave W_{SUM} stored last time) is used to determine the value of the local sound velocity in the region of interest.

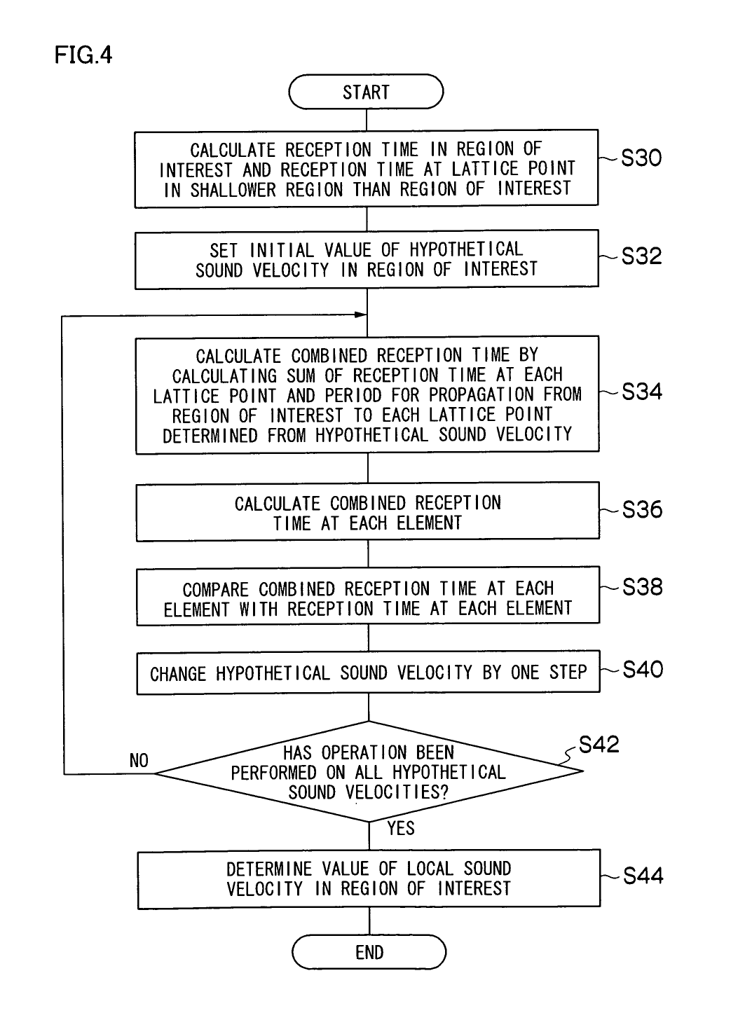

Next, a second embodiment is described, with reference to the flowchart illustrated in FIG. 4. The second embodiment enables a measurement of a local sound velocity even in where the sound velocity in the object is not uniform, and the reception time (or reception wave) of each of the elements corresponding to the respective lattice points cannot be approximated by the ambient sound velocity.

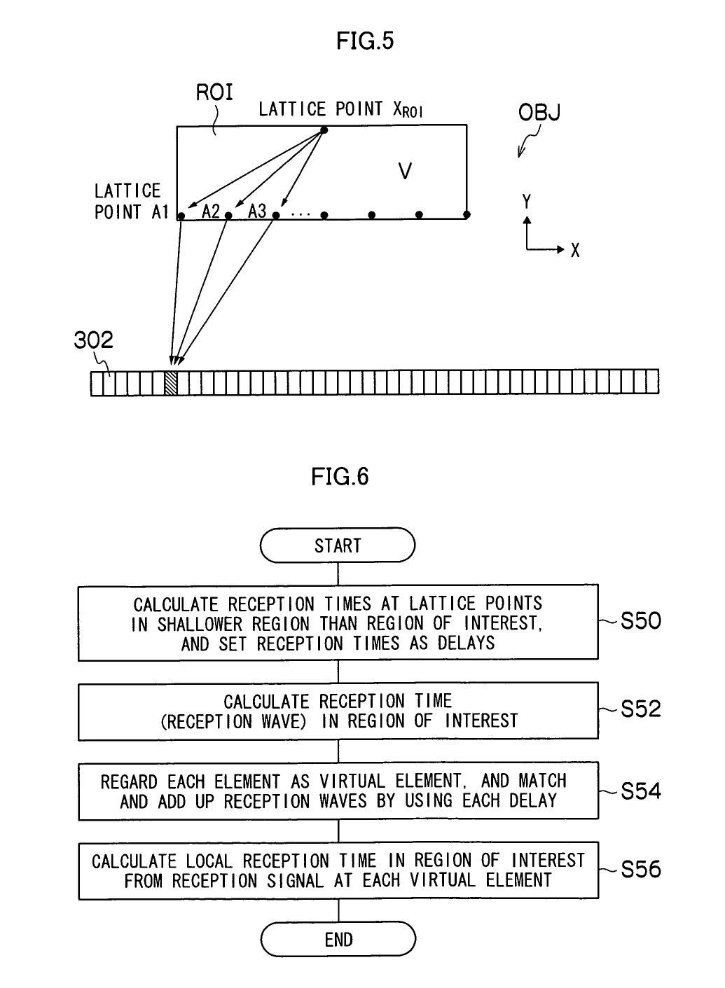

FIG. 5 schematically illustrates an operation to calculate the value of a local sound velocity according to this embodiment.

In FIG. 5, a representative lattice point in the region of interest ROI in the object OBJ is represented by X_{ROI}, and the lattice points arranged at regular intervals in the X-Y direction in shallower positions than the lattice point X_{ROI} (or in positions close to the ultrasonic transducers 302, or in a region between the lattice point X_{ROI} and the ultrasonic transducer 302) are represented by A1, A2, A3, . . . , as in FIG. 3. The sound velocities at least in a region between the lattice point X_{ROI} and the respective lattice points A1, A2, A3, . . . are assumed to be uniform. Here, the range of lattice points A1, A2, A3, . . . (a region where the lattice points A1, A2, A3, . . . are arranged) and the number of lattice points A1, A2, A3, . . . to be used in the operation to determine the value of the local sound velocity in the region of interest are determined in advance.

First, in step S30 of FIG. 4, the reception time in the region of interest and the reception times at the lattice points A1, A2, A3, . . . located in a shallower region than the region of interest are calculated. The reception time in the region of interest or at the lattice point X_{ROI}, and the reception times at the respective lattice points A1, A2, and A3, . . . can be calculated by a known image analysis method and a known phase aberration analysis method, as in the first embodiment.

In step S32, the initial value of the hypothetical sound velocity in the region of interest is set. In step S34, the periods for propagation from the region of interest to the respective lattice points which are determined by hypothetical sound velocities and the respective lattice point reception times are added up to calculate a combined reception time.

That is, the periods for propagation from the region of interest (the lattice point X_{ROI}) to the respective lattice points A1, A2, A3, . . . are calculated based on hypothetical sound velocities set in the region of interest ROI, and the sum of the propagation periods of time and the already determined reception times at the respective lattice points A1, A2, A3, . . . is calculated.

Where the hypothetical sound velocity in the region of interest ROI is represented by V, the periods for propagation from the lattice point X_{ROI} to the respective lattice points A1, A2, A3, . . . are calculated from X_{ROI}A1/V, X_{ROI}A2/V, X_{ROI}A3/V, . . . , respectively. Here, X_{ROI}A1, X_{ROI}A2, X_{ROI}A3, . . . represent the distances from the lattice point X_{ROI} to the respective lattice points A1, A2, A3, . . . , respectively, and can be calculated to be √(ΔX_{A1}²+ΔY²), √(ΔX_{A2}²+ΔY²), √(ΔX_{A3}²+ΔY²), . . . from the depth-direction width ΔY of the region of interest ROI and the X-direction distances ΔX_{A1}, ΔX_{A2}, ΔX_{A3}, . . . between the lattice point X_{ROI} and the respective lattice points A1, A2, A3, . . . , according to the Pythagorean theorem. Meanwhile, ΔY can be calculated to be ΔY=ΔT*V/2 from V and the depth-direction time width ΔT of the region of interest ROI (the difference between the reception time at the lattice point X_{ROI} and the reception time at the element immediately below the lattice point An located immediately below the lattice point X_{ROI} or the shortest reception time).

Since the reception times at the respective lattice points A1, A2, A3, . . . have already been calculated, the sums of the periods for propagation from the region of interest to the respective lattice points and the respective lattice point reception times can be calculated to obtain combined reception times.

In step S36, for each of the elements, the shortest combined reception time of the above combined reception times is set as the combined reception time at the element (that is, the path which minimizes the period for propagation is determined among the paths extending from the lattice point X_{ROI} to the element via a lattice point Aₖ (deflecting at Aₖ)).

In step S38, the above determined combined reception time (corresponding to the path which minimizes the period for propagation) of each element is compared with the reception time at the element from the lattice point X_{ROI}. The combined reception time at each element is compared with the reception time at the element, to determine the difference. The combined reception time with a smaller difference than that in the previous comparison (or the hypothetical sound velocity corresponding to this combined reception time) is stored.

In step S40, the hypothetical sound velocity is changed by one step. In step S42, a determination is made to determine whether the operation has been performed on all the hypothetical sound velocities. It should be noted that the range of hypothetical sound velocities to be subjected to the operation is set in advance.

If the operation has not been performed on all the hypothetical sound velocities, the operation returns to step S34, and the above described procedures are repeated.

If the operation has been performed on all the hypothetical sound velocities, the operation moves on to step S44, and the local sound velocity in the region of interest is determined from the combined reception time stored last time (or the hypothetical sound velocity corresponding to this combined reception time).

In the first and second embodiments, the local sound velocity in the region of interest can be calculated with higher precision by obtaining the total sum of errors of reception waves or reception times determined not at only one lattice point but at two or more lattice points arranged in the depth direction in the region of interest or two-dimensionally (or three-dimensionally). At this point, the mean local sound velocity of each lattice point may be determined, instead of the errors of the reception waves or reception times obtained at the lattice points.

Lattice points in a shallower region than the region of interest may not be set by the technique illustrated in FIGS. 3 and 5. For example, lattice points may be set on any curved surface located lower than the region of interest (or located on the side of the ultrasonic transducers 302). For example, lattice points may be set on the boundary with a tissue or lesion.

Next, a third embodiment of the presently disclosed subject matter is described. The third embodiment enables a calculation of a local reception time, instead of a local sound velocity, even where the sound velocity in the region of interest is not uniform.

FIG. 6 is a flowchart illustrating the operation flow according to the third embodiment.

In this embodiment, a representative lattice point X_{ROI} in the region of interest ROI in the object OBJ and shallower lattice points A1, A2, A3, . . . than the lattice point X_{ROI} are set, as in the above described embodiments as illustrated in FIG. 3, for example. The sound velocities at least between the lattice point X_{ROI} and the respective lattice points A1, A2, A3, . . . are assumed to be uniform.

First, in step S50 of FIG. 6, reception times at the respective lattice points A1, A2, A3, . . . located in a shallower region than the region of interest are calculated by an image analysis method and a phase aberration analysis method. The reception times are then set as delays at the respective lattice points A1, A2, A3, . . .

In step S52, the reception time (or reception wave) in the region of interest (the lattice point X_{ROI}) is also calculated by an image analysis method and a phase aberration analysis method.

It should be noted that the order of step S50 and step S52 may be reversed, and the reception time in the region of interest may be calculated first.

In step S54, the respective lattice points A1, A2, A3, . . . are regarded as virtual elements. And, reception signals of the respective virtual elements are signals obtained by matching and adding up reception waves in the region of interest with the use of delays which are the above calculated reception times at the respective lattice points Al, A2, A3, . . .

In step S56, the local reception time of the region of interest is calculated by carrying out a phase aberration analysis on the reception signal of each of the virtual elements.

After the local reception time of the region of interest is calculated, the local sound velocity can be determined from the local reception time.

Next, a fourth embodiment of the presently disclosed subject matter is described. The fourth embodiment is another embodiment which enables a calculation of a local reception time, instead of a local sound velocity, even where the sound velocity in the region of interest is not uniform.

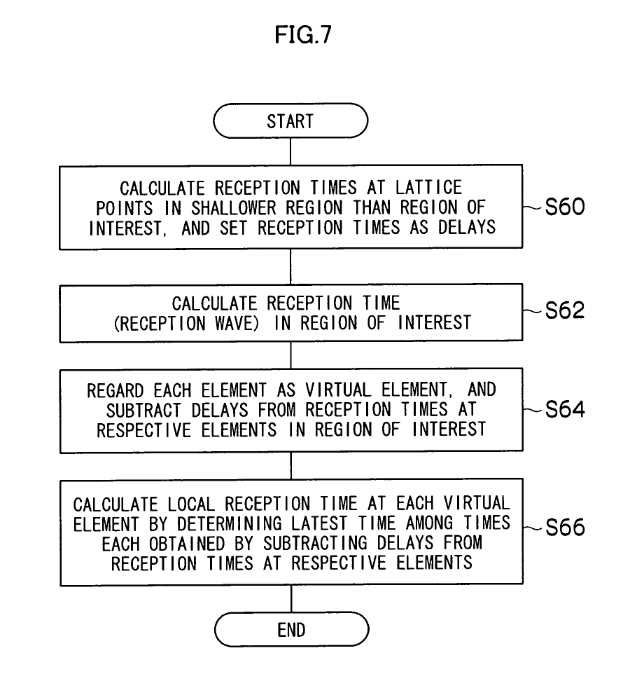

FIG. 7 is a flowchart illustrating the operation flow according to the fourth embodiment.

In this embodiment, a representative lattice point X_{ROI} in the region of interest ROI in the object OBJ and shallower lattice points A1, A2, A3, . . . than the lattice point X_{ROI} are set, as in the above described embodiments as illustrated in FIG. 3, for example. The sound velocities at least between the lattice point X_{ROI} and the respective lattice points A1, A2, A3, . . . are assumed to be uniform.

First, in step S60 of FIG. 7, reception times at the respective lattice points A1, A2, A3, . . . located in a shallower region than the region of interest ROI are calculated by an image analysis method and a phase aberration analysis method. The reception times are then set as delays at the respective lattice points A1, A2, A3, . . .

In step S62, the reception time (or reception wave) in the region of interest (the lattice point X_{ROI}) is also calculated by an image analysis method and a phase aberration analysis method.

It should be noted that the order of step S60 and step S62 may be reversed, and the reception time in the region of interest may be calculated first.

In step S64, the respective lattice points A1, A2, A3, . . . are regarded as virtual elements, and the delays are subtracted from the reception times of the respective elements in the region of interest.

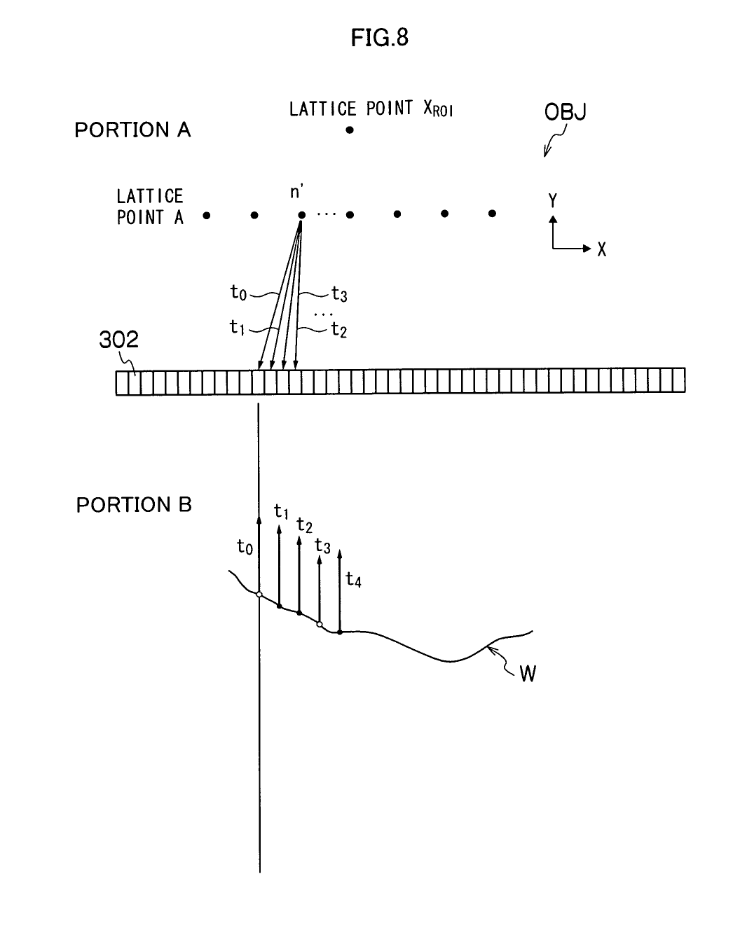

FIG. 8 schematically illustrates this situation.

In FIG. 8, the respective lattice points A (A1, A2, A3, . . . ) are regarded as virtual elements, and the reception times of reception waves from a virtual element n′ to the respective ultrasonic transducers 302 are represented by t0, t1, t2, t3, . . . On the other hand, W illustrated in the lower portion of FIG. 8 represents an actual reception wave from the lattice point X_{ROI}, and the respective reception times t0, t1, t2, t3, . . . as delays are subtracted from the reception wave W.

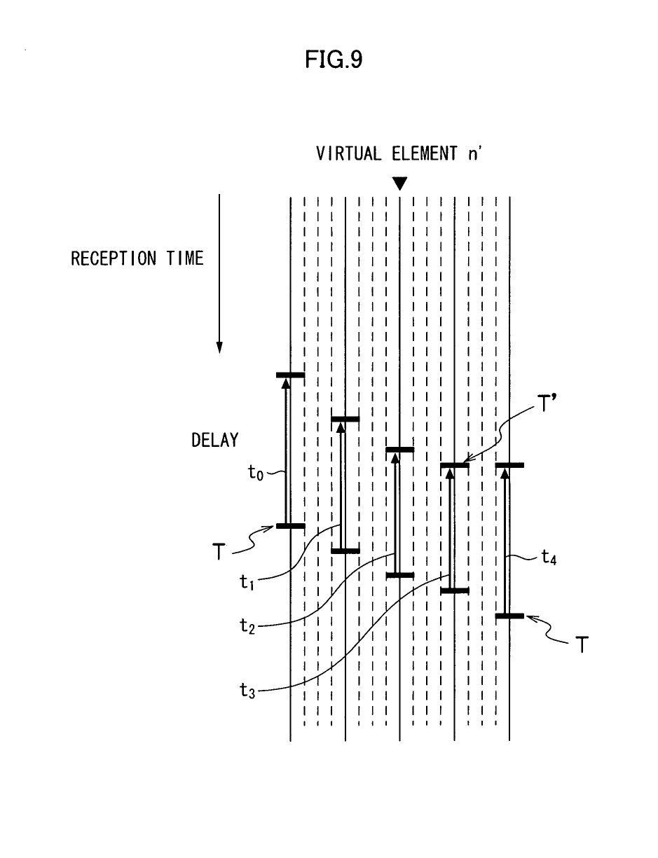

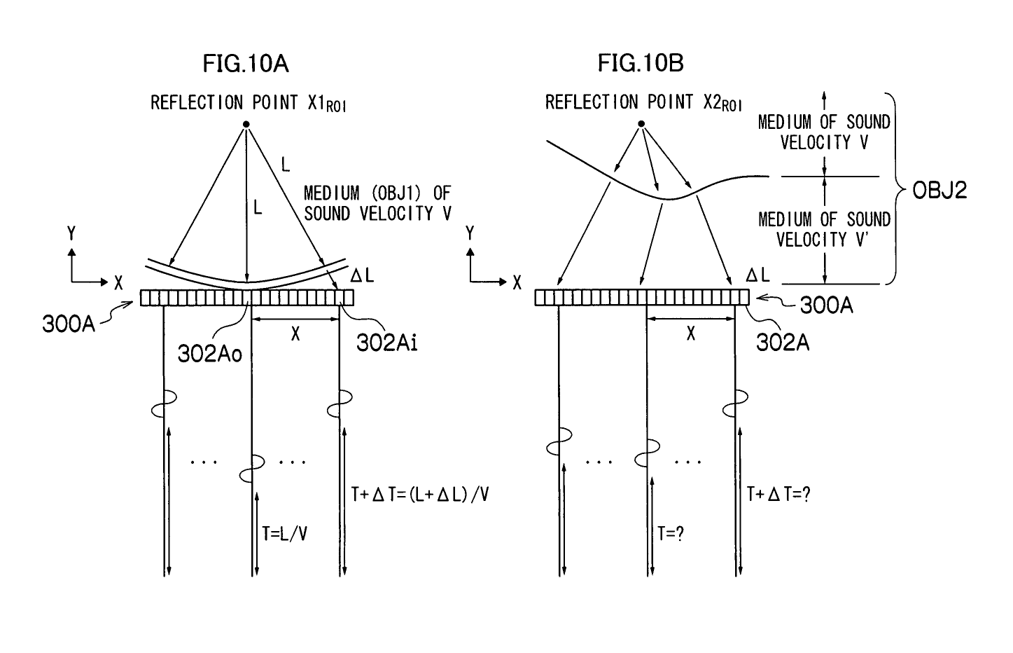

FIG. 9 illustrates a situation where the delays are subtracted from the reception wave.

In FIG. 9, the solid lines indicate the respective elements, and the broken lines indicate the virtual elements. The reception time becomes longer toward the bottom side of the drawing. Since the downward direction in the drawing is the direction in which the reception time increases, the subtractions of the delays t0, t1, t2, t3, . . . are indicated by the upward arrows. Here, the bases of the arrows indicating the respective delays t0, t1, t2, t3, . . . represent the reception times T in the region of interest.

In step S66 of FIG. 7, the latest time of the times obtained by subtracting the delays from the reception times of the respective elements are set as the local reception times of the respective virtual elements.

Since the downward direction is the direction in which the reception time increases in FIG. 9, the top end of the arrow located in the lowest position among the top ends of the arrows representing the subtractions of delays represent the latest time. That is, the time T′ from which the delay t3 has been subtracted is the latest time in FIG. 9, and the time T′ is employed as the local reception time at the virtual element n′.

As described above, instead of a local sound velocity, a local reception time can also be calculated in this embodiment. After the local reception time in the region of interest is determined, the local sound velocity can be calculated from the local reception time.

In a case where all the reception waves (reception times) at the respective lattice points A1, A2, A3, . . . located in a shallower region than the region of interest can be regarded as the same reception wave W_{A}, a local reception time in the region of interest can be calculated by the following method. The reception wave in the region of interest (at the lattice point X_{ROI}) can be regarded as a result obtained by delaying the reception wave W_{A} by the periods for propagation from the lattice point X_{ROI} to the respective lattice points A1, A2, A3, . . . , followed by convolutions. Accordingly, the periods for propagation from the lattice point X_{ROI} to the respective lattice points A1, A2, A3, . . . (local reception times) can be calculated by deconvoluting the reception wave at the lattice point X_{ROI} with the reception wave W_{A}. The deconvoluting process can be performed on the reception signal of each element or on the frequency space.

Alternatively, the periods for propagation from the lattice point X_{ROI} to the respective lattice points A1, A2, A3, . . . may be set so that the difference between the reception wave (reception time) at the lattice point X_{ROI} and a reception wave (reception time) at the lattice point X_{ROI} calculated from the reception waves (reception times) at the lattice points A1, A2, A3, . . . and the periods for propagation from the lattice point X_{ROI} to the respective lattice points A1, A2, A3, . . . is minimized. There are various kinds of least-value search algorithms, but the quasi-Newton's method may be used, for example.

As described above, in the first and second embodiments of the presently disclosed subject matter, instead of the ambient sound velocity, a reception time is calculated and held at each lattice point. In the third and fourth embodiments, instead of a local sound velocity in the region of interest, a local reception time is calculated. In any case, a local sound velocity can be calculated with high precision even where the sound velocity in the object is not uniform, and the reception time at each lattice point cannot be approximated by the ambient sound velocity.

Although ultrasonic diagnosis apparatuses and ultrasonic diagnosis methods according to the presently disclosed subject matter have been described so far, the presently disclosed subject matter is not limited to the above examples, and various changes and modifications may of course be made to them without departing from the scope of the invention.

What is claimed is:

1. An ultrasonic diagnosis apparatus comprising:
an ultrasonic probe which includes a plurality of ultrasonic transducers configured to transmit ultrasonic waves to an object, to receive the ultrasonic waves reflected by the object, and to output ultrasonic detection signals;
a reception time calculating device configured to calculate at least one of a reception time and a reception wave at each of respective elements corresponding to two or more lattice points at different depths in the object with respect to the ultrasonic probe; and
a local sound velocity calculating device configured to calculate a local sound velocity which is a sound velocity in a local region, using calculated one of the reception time and the reception wave.

2. The ultrasonic diagnosis apparatus according to claim 1, wherein
the two or more lattice points at different depths with respect to the ultrasonic probe include a lattice point in a region of interest in the object and a lattice point which is set in a shallower region than the region of interest.

3. The ultrasonic diagnosis apparatus according to claim 2, wherein
the local sound velocity calculating device calculates the local sound velocity by comparing a reception wave at the region of interest with a resultant reception wave formed by combining a reception wave at the lattice point set in the shallower region with a delay determined by a hypothetical sound velocity hypothetically set in the region of interest.

4. The ultrasonic diagnosis apparatus according to claim 2, wherein
the local sound velocity calculating device calculates the local sound velocity by comparing a reception time at the lattice point of the region of interest with a smallest value of sums, each of the sums being a sum of a propagation time determined based on a hypothetical sound velocity hypothetically set in the region of interest and the reception time at the lattice point set in the shallower region, the propagation time being a period for propagation from the lattice point in the region of interest to the lattice point set in the shallower region.

5. An ultrasonic diagnosis apparatus comprising:
an ultrasonic probe which includes a plurality of ultrasonic transducers configured to transmit ultrasonic waves to an object, to receive the ultrasonic waves reflected by the object, and to output ultrasonic detection signals;
a reception time calculating device configured to calculate at least one of a reception time and a reception wave at each of respective elements corresponding to two or more lattice points at different depths in the object with respect to the ultrasonic probe; and
a local reception time calculating device configured to calculate a local reception time which is a reception time in a local region, using calculated one of the reception time and the reception wave.

6. The ultrasonic diagnosis apparatus according to claim 5, wherein
the two or more lattice points at different depths with respect to the ultrasonic probe include a lattice point in a region of interest in the object and a lattice point set in a shallower region than the region of interest.

7. The ultrasonic diagnosis apparatus according to claim 6, wherein
the local reception time calculating device calculates a local reception time by regarding the lattice point set in the shallower region as a virtual element, matching and adding up a reception wave at the lattice point in the region of interest and a delay which is a reception time at the lattice point set in the shallower region, and carrying out a phase aberration analysis on a reception signal of the virtual element.

8. The ultrasonic diagnosis apparatus according to claim 6, wherein
the local reception time calculating device regards the lattice point set in the shallower region as a virtual element, and calculates a local reception time at the virtual element, a local reception time being a latest time among times each obtained by subtracting a delay which is a reception time at the lattice point set in the shallower portion from the reception time at the lattice point in the region of interest.

9. An ultrasonic diagnosis method comprising:
transmitting ultrasonic waves to an object through an ultrasonic probe including a plurality of ultrasonic transducers, and outputting ultrasonic detection signals after receiving the ultrasonic waves reflected by the object;
calculating at least one of a reception time and a reception wave at each of respective elements corresponding to two or more lattice points at different depths in the object with respect to the ultrasonic probe; and
calculating a local sound velocity which is a sound velocity in a local region, using calculated one of the reception time and the reception wave.

10. The ultrasonic diagnosis method according to claim 9, wherein
the calculating the reception time includes using an image analysis and a phase aberration analysis to calculate the reception time.

11. The ultrasonic diagnosis method according to claim 9, wherein
the two or more lattice points at different depths with respect to the ultrasonic probe include a lattice point in a region of interest in the object and a lattice point set in a shallower region than the region of interest.

12. The ultrasonic diagnosis method according to claim 11, wherein
the calculating the local sound velocity includes comparing a reception wave at the region of interest with a resultant reception wave formed by combining a reception wave at the lattice point set in the shallower region with a delay which is determined by a hypothetical sound velocity hypothetically set in the region of interest.

13. The ultrasonic diagnosis method according to claim 11, wherein
the calculating the local sound velocity includes comparing a reception time at the lattice point in the region of interest with a smallest value of sums, each of the sums being a sum of a propagation time determined based on a hypothetical sound velocity hypothetically set in the region of interest and a reception time at the lattice point set in the shallower region, the propagation time being a period for propagation from the lattice point in the region of interest to the lattice point set in the shallower region.

14. An ultrasonic diagnosis method comprising:
transmitting ultrasonic waves to an object through an ultrasonic probe including a plurality of ultrasonic transducers, and outputting ultrasonic detection signals after receiving the ultrasonic waves reflected by the object;
calculating at least one of a reception time and a reception wave at each of respective elements corresponding to two or more lattice points at different depths in the object with respect to the ultrasonic probe; and
calculating a local reception time which is a reception time in a local region, using calculated one of the reception time and the reception wave.

15. The ultrasonic diagnosis method according to claim 14, wherein
the calculating the reception time includes using an image analysis and a phase aberration analysis to calculate the reception time.

16. The ultrasonic diagnosis method according to claim 14, wherein
the two or more lattice points at different depths with respect to the ultrasonic probe include a lattice point in a region of interest in the object and a lattice point which is set in a shallower region than the region of interest.

17. The ultrasonic diagnosis method according to claim 16, wherein
the calculating the local reception time includes regarding the lattice point set in the shallower region as a virtual element, matching and adding up a reception wave at the lattice point in the region of interest and a delay which is a reception time at the lattice point set in the shallower region, and carrying out a phase aberration analysis on a reception signal of the virtual element.

18. The ultrasonic diagnosis method according to claim 16, wherein
the calculating the local reception time includes regarding the lattice point set in the shallower region as a virtual element, and calculating a local reception time at the virtual element, a local reception time being a latest time among times each obtained by subtracting a delay which is a reception time at the lattice point set in the shallower portion from a reception time at the lattice point in the region of interest.
